(19) United States
(12) Patent Application Publication
(10) Pub. No.: US 2011/0151462 A1
(43) Pub. Date: Jun. 23, 2011

(54) BACTERIAL VAGINOSIS APPARATUS AND DIAGNOSTIC METHODS

(76) Inventors: Katherine Tynan, Mountainview, CA (US); Gary Schoolmik, Mountainview, CA (US)

(21) Appl. No.: 12/677,144

(22) PCT Filed: Sep. 10, 2008
(86) PCT No.: PCT/US08/75781

§ 371 Date: Feb. 8, 2011

Related U.S. Application Data
(60) Provisional application No. 60/971,230, filed on Sep. 10, 2007.

(51) Int. Cl. C12Q 1/06; C12Q 1/68

(52) U.S. Cl. 435/6.12; 435/39

(57) ABSTRACT

The present disclosure relates to the filed of medical diagnostics, specifically directed towards the field of bacterial vaginosis diagnostic methods, systems and apparatus. Also included is a multiplexed platform test for the diagnosis of infectious vaginitis.

### TECHNICAL FIELD

The present invention relates generally to the field of medical diagnostics. More specifically, the present invention is directed toward the field of bacterial vaginosis diagnostic methods, systems and apparatus.

### BACKGROUND

The normal vaginal ecosystem is complex, predominated by Lactobacilli. Lactobacillus is estrogen dependent, maintaining a normal environmental pH of 4.0. Lactobacillus preserves mucin gel coating over the epithelium, produces lactic acid, H₂0₂, and bacteriocins (calprotectin), and maintains an innate immune response. Lactobacillus protects against HIV acquisition/transmission, prevents pro-inflammatory cytokines and reduces the risk of STIs (sexually transmitted infections). (Valore, Am J Obstet Gynecol; 87: 561 (2002).)

A change in normal bacterial flora, including the reduction of Lactobacillus allows other bacteria to multiply and produce toxins which affect the body's natural defenses and make re-colonization of healthy bacteria more difficult. Normal vaginal flora is commonly described in the literature. Specifically normal vaginal flora is described by: Fredricks, et al., Molecular identification of bacteria associated with bacterial vaginosis. N. Engl. J. Med. 353:1899-1911. 2005; Hyman, et al., Microbes on the human vaginal epithelium. PNAS 120:7952-7957, 2005; Verhelst, et al., Cloning of 16S rRNA genes amplified from normal and disturbed vaginal microflora suggests a strong association between Atopobium vaginae, Gardnerella vaginalis and bacterial vaginosis. BMC Microbiol. 4:16. 2004; Verhelst, et al., Comparison between Gram stain and culture for the characterization of vaginal microflora: definition of a distinct grade that resembles grade I microflora and revised categorization of grade I microflora. BMC Microbiol. 5:61. 2005; Zhou, et al., Characterization of vaginal microbial communities in adult healthy women using cultivation-independent methods. Microbiology 150: 2565-2573. 2004; May et al., The Identification of Vaginal Lactobacillus Species and the Demographic and Microbiologic Characteristics of Women Colonized by These Species. The Journal of Infectious Diseases 180:1950-6. 1999; and Vasquez et al., Vaginal Lactobacillus Flora of Healthy Swedish Women. Journal of Clinical Microbiology, 40 (8). 2746-2749. 2002.

Infectious vaginitis accounts for more than ten million physician office visits each year, with approximately half of all adult women suffering at least one episode. The three most common forms of infectious vaginitis in decreasing incidence are bacterial vaginosis, vulvovaginal candidiasis and trichomoniasis. Because vulvovaginal infections can result in serious clinical sequelae, vulvovaginal symptoms and signs warrant careful evaluation and appropriate therapy.

Bacterial vaginosis (BV) is a prevalent cause of vaginal infection and is associated with several serious health conditions. BV is caused by an imbalance of naturally occurring bacterial flora. BV results when the normal, predominantly Lactobacillus, vaginal flora shifts to one dominated by Gardnerella vaginalis, Mycoplasma hominis, and a variety of anaerobic organisms predominantly clostridial species.

Fredricks, et al. have reported considerable bacterial diversity in subjects who had BV as compared to healthy subjects, including 35 bacterial phylotypes detected in samples from subjects with BV. (Fredricks, et al. “Molecular Identification of Bacteria Associated with Bacterial Vaginosis” N. Engl. J. Med. 353;18 (2005).) Additionally, it was reported that extremely high levels of bacterial DNA from several rarely cultivated clostridial species were detected in the vaginal fluid of subjects with BV. Fielder, et al. “Changes in BV-Associated Bacterial concentrations with Vaginal Metronicazole Therapy” ISSTDR abstract (2007)).

The pathogenesis of BV is as follows: the overgrowth of anaerobic microorganisms is accompanied by the production of proteolytic enzymes that act on vaginal peptides to release several biologic products, including polyamines, which volatize in the accompanying alkaline environment to elaborate foul-smelling trimethylamine. Polyamines facilitate the transudation of vaginal fluid and exfoliation of epithelial cells, creating a copious discharge. Clue cells are formed when such organisms are present in high numbers, adhere to exfoliated epithelial cells in the presence of an elevated pH. (Sobel, J D, N Engl. J. Med. 337: 1896-1903 (1997).)

Untreated BV may cause serious complications, such as increased susceptibility to sexually transmitted infections and may present other complications for pregnant women. BV has also been associated with an increase in the development of Pelvic Inflammatory Disease following surgical procedures such as a hysterectomy or an abortion. BV can be cured by antibiotics such as metronidazole and clindimicyn. Thus it is important to have an effective efficient method for diagnosis.

Prior BV diagnostics focus on causative agents, either applying direct or indirect measures. These diagnostics include detection of Gardnerella vaginalis through a nucleic acid based test. Additionally, prior diagnosis of BV includes detection of elevated enzymes such as sialidase activity, an enzyme produced by bacterial pathogens associated with BV including Gardnerella, Bacteroides, Prevotella and Mobilincus.

All of the references cited in this disclosure are hereby incorporated in their entirety.

### SUMMARY OF THE DISCLOSURE

The present disclosure teaches a method for the diagnosis of BV through a quantitative determination of Lactobacillus, establishing a measure of clinically relevant Lactobacillus in healthy vaginas, and correlating a diminished amount of Lactobacillus with the diagnosis of BV. The present disclosure also teaches a method for detecting the occurrence or non-occurrence of bacterial vaginosis in a patient comprising the steps of collecting a biological sample from a patient, quantitatively measuring the presence of Lactobacillus; and correlating a diminished presence of Lactobacillus with the diagnosis of bacterial vaginosis. The disclosure further teaches using quantitative polymerase chain reaction to Lactobacillus in order to quantitate the amount of Lactobacillus. The disclosure further teaches using any analyte sandwich assay (i.e. lateral flow immunoassay, ELISA or direct DNA detection) to quantitate the amount of Lactobacillus. The disclosure further teaches a method for diagnosis of BV, Candida and Trichomonas utilizing lateral flow immunoassay to quantitate Candida, Trichomonas and Lactobacillus in a multiplexed assay.

### BRIEF DESCRIPTION OF THE FIGURES

FIG. 1 is a graphic representation of an inverse relationship between Lactobacillus crispatus and BV pathogens.

FIG. 2 is a graphic representation of changes in BV flora following Metronidazole treatment.

FIG. 3 is a flow chart representation of a syndromic approach to diagnosis of BV, Candida and Trichomonas, collectively called vaginosis.

FIG. 4 is a flow chart representation of an additional step in the syndromic approach to diagnosis of BV, Candida and Trichomonas, collectively called vaginosis.

### DETAILED DESCRIPTION OF THE INVENTION

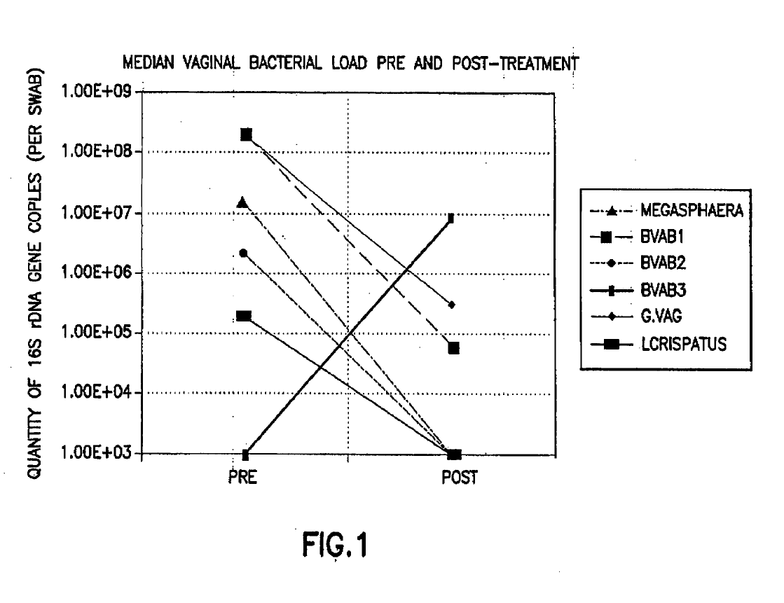

Lactobacillus crispatus, is the predominant Lactobacillus species present in “healthy vaginas” as shown in the control subjects of Table 1. Table 1 includes the results of a study of the bacteria identified by broad-range 165 rDNA polymerase chain reaction in vaginal fluid from subjects with bacterial vaginosis. (Fredricks et al. N Eng J Med 2005) There are a large and diverse number of bacterial species found in the vagina of subjects with BV. FIG. 1 illustrates the existence of an inverse relationship between Lactobacillus crispatus and BV pathogens in subjects with BV. Thus the level of Lactobacillus crispatus significantly decreases with a diagnosis of BV.

TABLE 1
Bacteria Identified by Broad-Range 16SrDNA Polymerase Chain Reaction in
Vaginal fluid from Subjects with and without Bacterial Vaginosis.

| | Subjects with BV | | | | | | | | | Control Subjects without BV | | | | | | | |
|---|---|---|---|---|---|---|---|---|---|---|---|---|---|---|---|---|---|
| | 1 | 2 | 3 | 4 | 5 | 6 | 7 | 8 | 9 | 1 | 2 | 3 | 4 | 5 | 6 | 7 | 8 |
| | | | | | | | Percentage of Clones | | | | | | | | | | |
| Lactobacillus crispatus | | | | | | | | | | 49 | 74 | 99 | 48 | 60 | 89 | 40 | 100 |
| L. jensenii | | | | | | | | | | | | | 2 | | 1 | | |
| L. gallinarum | | | | | | | | | | 13 | | | | | | | |
| L. gasseri | | | | | | | | | | | 9 | | | 1 | | | |
| L. vaginalis | | | | | | | | | | | | | 2 | | | | |
| Staphylococcus | | | | | | | | | | | 2 | | | | | | |
| epidermidis | | | | | | | | | | | | | | | | | |
| S. lugdunensis | | | | | | | | | | | | | 1 | | | | |
| Clostridium | | | | | | | | | | | | | | 3 | | | |
| perfringens (96%) | | | | | | | | | | | | | | | | | |
| AB045286 | | | | | | | | | | | | | | | | | |
| Ureaplasma parvum | | | | | | | | | | | 1 | | | | | | |
| L. iners | | | | 7 | 22 | 3 | | 1 | 5 | 38 | | 1 | 46 | 36 | 10 | 60 | |
| Gardnerella vaginalis | 35 | 13 | 2 | 4 | 28 | 1 | 25 | 31 | 39 | | 14 | | | | | | |
| Uncultured AB034121 | | 43 | 66 | 34 | | 36 | 17 | | 26 | | | | | | | | |
| (90.6%) (BVAB1) | | | | | | | | | | | | | | | | | |
| Uncultured AF407407 | 10 | 4 | | 10 | 3 | 5 | 4 | | 4 | | | | | | | | |
| (90.9%) (BVAB2) | | | | | | | | | | | | | | | | | |
| Uncultured | 1 | 1 | | | | | 1 | | | | | | | | | | |
| ULO278163 (92.9%) | | | | | | | | | | | | | | | | | |
| (BVAB3) | | | | | | | | | | | | | | | | | |
| Atopobium vaginae | 5 | | 3 | 21 | 1 | 3 | 3 | 11 | 11 | | | | | | | | |
| Leptotrichia amnionii | 6 | | 2 | 8 | 1 | 3 | 10 | 10 | | | | | | | | | |
| Megasphaera elsdenii | 4 | 10 | 7 | 1 | 18 | 3 | 2 | 13 | 6 | | | | | | | | |
| (93.8%) AY038994 | | | | | | | | | | | | | | | | | |
| M. micronuciformis | | | | | | | 1 | | | | | | | | | | |
| (94.6%) AF473833 | | | | | | | | | | | | | | | | | |
| Eggerthella | 2 | 2 | 2 | 1 | 4 | 1 | 3 | 8 | | | | | | | | | |
| hongkongensis | | | | | | | | | | | | | | | | | |
| (91.8%) AY288517 | | | | | | | | | | | | | | | | | |
| Porphyromonas | | | | 2 | | 5 | | | | | | | | | | | |
| asaccharolytica | | | | | | | | | | | | | | | | | |
| Dialister species (a) | 1 | 1 | 2 | 2 | | | | | | | | | 1 | | | | |
| Dialister species (b) | | 1 | 2 | | | 2 | 2 | 1 | | | | | | | | | |
| (94.8%) AF371693 | | | | | | | | | | | | | | | | | |
| Sneathia sanguinegens | 3 | | 2 | | 16 | 9 | 9 | | | | | | | | | | |
| Prevotella genogroup | 21 | 24 | | 1 | 7 | 9 | 12 | 20 | | | | | | | | | |
| 1 | | | | | | | | | | | | | | | | | |
| Prevotella genogroup | 7 | | | 4 | | 6 | 7 | | 1 | | | | | | | | |
| 2 | | | | | | | | | | | | | | | | | |
| Prevotella genogroup | | | 7 | | | | | 3 | | | | | | | | | |
| 3 | | | | | | | | | | | | | | | | | |
| P. bivia | | | | | | | | | | | | | | | | | |
| P. buccalis | | | | | | | | | 6 | | | | | | | | |
| P. dentalis (93.2%) | | | | | | 2 | 2 | | | | | | | | | | |
| X81876 | | | | | | | | | | | | | | | | | |
| P. disiens | | | | | | | | | | | | | | | | | |
| P. oulorum (90.6%) | | | | | | | | | | | | | | | | | |
| L16472 | | | | | | | | | | | | | | | | | |
| P. shahii (90.7%) | | | | | | | | | | | | | | | | | |
| AB108825 | | | | | | | | | | | | | | | | | |
| Uncultured 4C28d-23 | 1 | | 3 | 1 | | 2 | | | | | | | | | | | |
| (91.2%) AB034149 | | | | | | | | | | | | | | | | | |
| Candidate division | | | 2 | 2 | | 8 | | | | | | | | | | | |
| TM7 (93.7%) | | | | | | | | | | | | | | | | | |
| AF125206 | | | | | | | | | | | | | | | | | |
| Mobiluncus mulieris | | | | | | | | | | | | | | | | | |
| Peptoniphilus | | | | | | | 1 | | | | | | | | | | |
| lacrimalis | | | | | | | | | | | | | | | | | |
| Peptoniphilus species | | 1 | | | | 2 | | | | | | | | | | | |
| Peptostreptococcus | 3 | | | 2 | | | | 1 | | | | | | | | | |
| micros (97.8%) | | | | | | | | | | | | | | | | | |
| AF542231 | | | | | | | | | | | | | | | | | |
| Gemella bergeriae | 1 | | | | | | | | | | | | | | | | |
| (95.8%) Y13365 | | | | | | | | | | | | | | | | | |
| Aerococcus species | | | | | | | | 1 | 2 | | | | | | | | |
| Anaerococcus | | | | | | | | | | | | | | | | | |
| tetradius | | | | | | | | | | | | | | | | | |
| Uncultured (89.8%) | | | | | | | | | | | | | | | | | |
| AF371910 | | | | | | | | | | | | | | | | | |
| Uncultured (88.4%) | | | | | | | | | | | | | | | | | |
| AJ400235 | | | | | | | | | | | | | | | | | |
| Veillonella species | | | | | | | 1 | | | | | | | | | | |

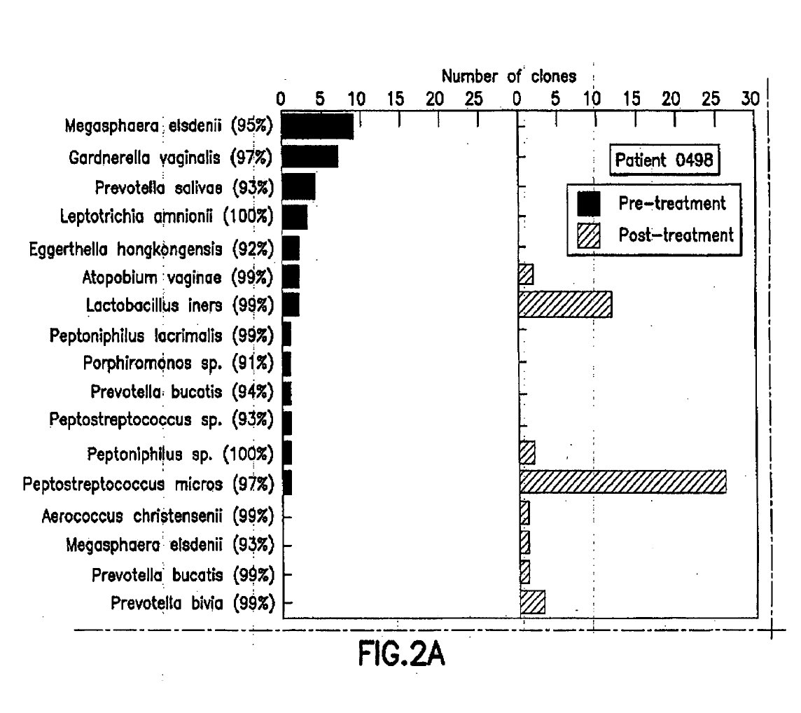

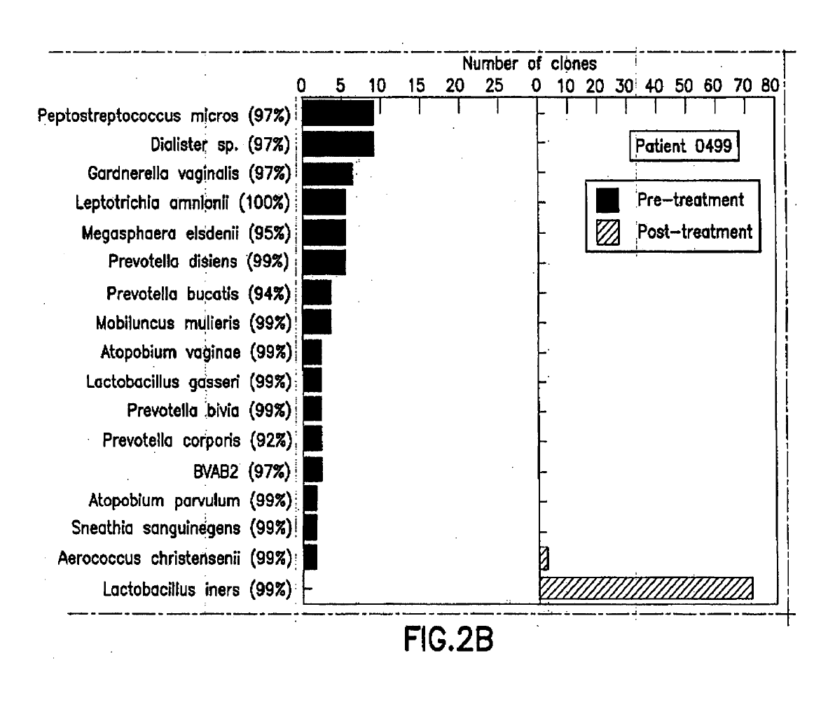

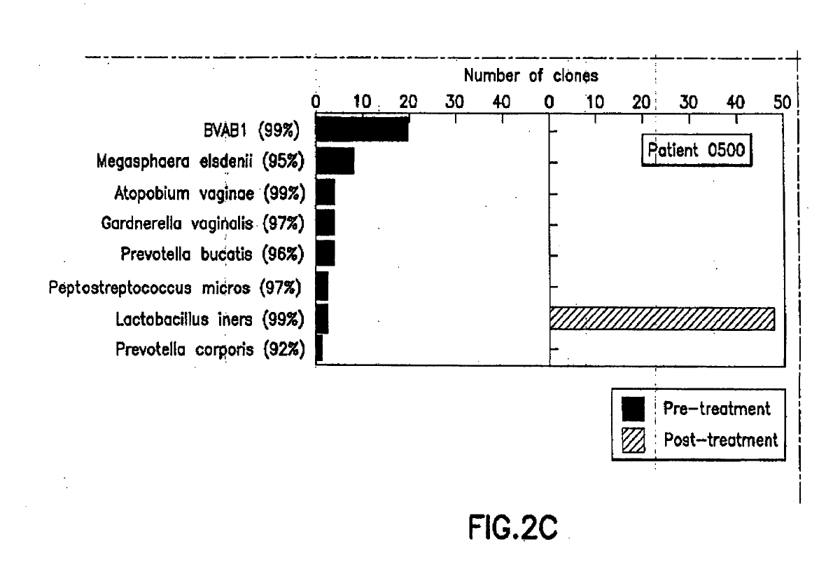

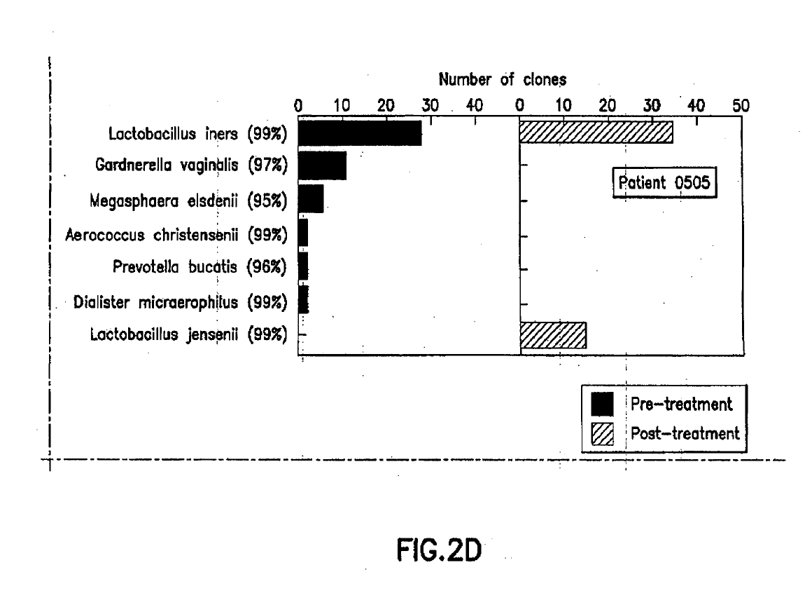

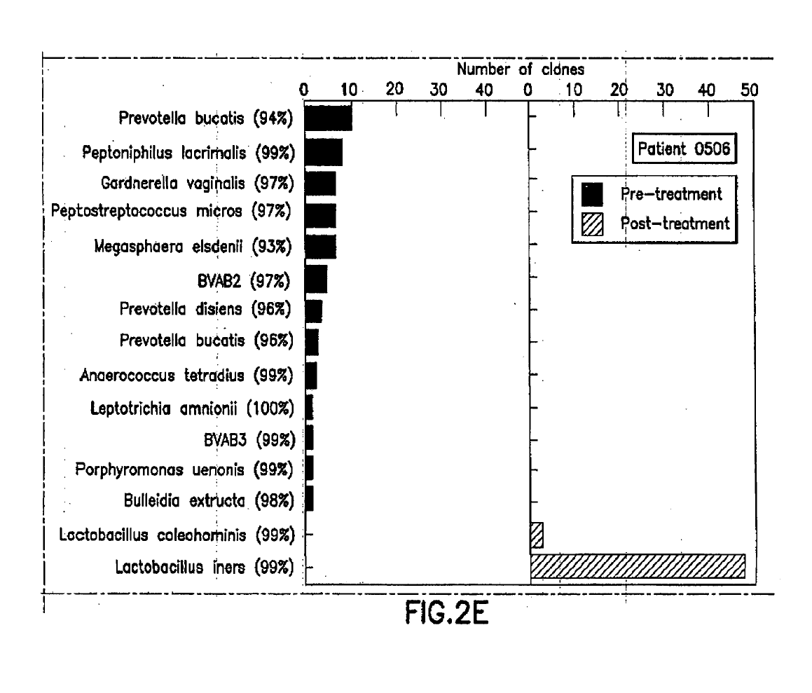

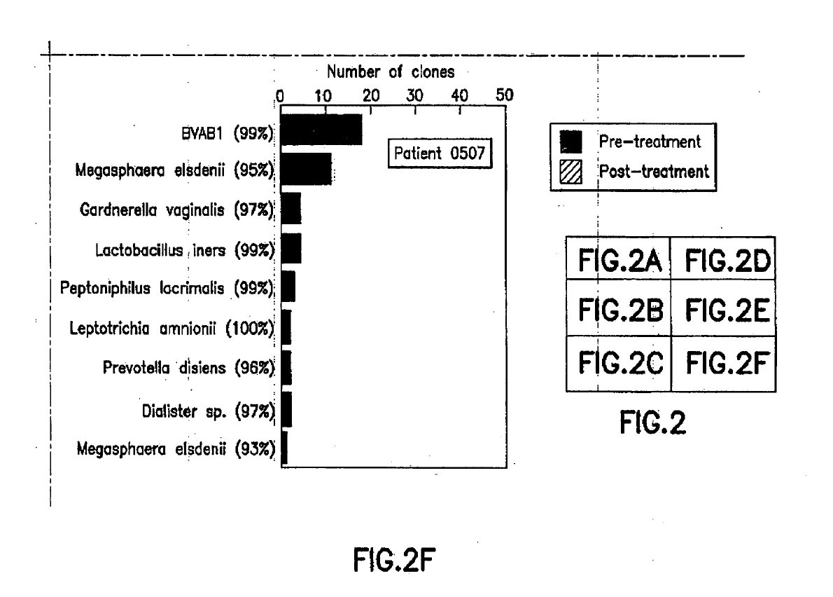

As shown in FIG. 2, Polymerase Chain Reaction (PCR) was used to survey BV flora before and after metronidazole treatment. (Ferris et al. J. of Clin. Microbiology, 2007). The species composition for patients prior to treatment was variable. Lactobacillus (in particular Lactobacillus inners) was prominent in all patients post-treatment. Atopobium vaginae concentrations were highest for patients who failed or responded incompletely to treatment and lowest for patients who were cured. Thus the results illustrated that post treatment, Lactobacillus predominate. Thus, a normal level of Lactobacillus present in the vaginal flora is keystone to a “healthy vagina”.

The present disclosure teaches a method for the diagnosis of BV. Quantitative measure of Lactobacillus as a measure of a healthy vagina and an inverse correlation with BV yields a diagnostic test for BV. This method differs from previous diagnostics where the presence of the multitude of agents associated with BV, such as Gardnerella, indirect enzyme measures, Amsel criteria etc., was identified for diagnosis. The present disclosure teaches quantitatively measuring clinically relevant Lactobacilli. These Lactobacilli include, but are not limited to, crispatus, jensenii, and gasseri, either independently or in combination. Quantitative measurement can be achieved by a variety of methods known in the art. These methods include, but are not limited to: antibody/homologous cross reactive but specific reagents, quantitative polymerase chain reaction, and any “sandwich” assay—both antibody and nucleic acid based. Further included are direct antigen measurement and lateral flow immunoassay including utilizing SERS technology. The present disclosure teaches establishing a clinically relevant cut off value for the Lactobacillus measure from clinical trials and the use of statistical measures of relevance (ROC curves (receiver operator curves) as would be understood by one of skill in the art.

Table 2 lists the analyte targets, antibodies for cytoplasmic proteins, antibodies for surface proteins and sequence information for applicable targets, including Candida, Trichomonas and Lactobacillus. Using the disclosed sequences, one with skill in the art can readily and without undue experimentation, develop antibodies to Candida, Trichomonas and Lactobacillus. As such, antibodies for Candida and Trichomonas are already known and listed in the table. Additionally, methods to develop antibodies using sequence derived antigens as described can be found at Strategic Diagnostics Inc, http://www.sdix.com/. In the case of Lactobacillus, the sequences disclosed and known are used to determine antigenic regions and will be further used to raise antibodies.

TABLE 2

| | | Antibody for | |
|---|---|---|---|
| Analyte | Antibody for cytoplasmic | surface | |
| Targets | protein | protein | Sequence information |
| Candida | PA1-7206 Polyclonal, | MA1-7009, | The completed assembly #21 contains 15.845 Mb of |
| albicans | ABR—Affinity BioReagents | Monoclonal, | DNA, organized into the 8 C. albicans chromosomes. |
| | ab53891 Polyclonal, Abcam | ABR—Affinity | The gene and protein sequences or the sequence of part |
| | Ab34211 monoclonal, Abcam | BioReagents | or all of a chromosome can be retrieved from the |
| | 1. monoclonal, | | Candida Genome |
| | AbD serotec | | Database.http://www.candidagenome.org/ |
| | 2. monoclonal, | | * The genomic sequence of Candida glabrata |
| | AbD serotec | | chromosomes A-M is available in Genebank. |
| | 3. monoclonal, | | http://www.ncbi.nlm.nih.gov/mapview/maps.cgi?taxid= |
| | AbD serotec | | 284593&chr=A |
| | 1750-5007 Polyclonal, | | |
| | AbD serotec | | |
| Trichomonas | 9155-9529 monoclonal, | C65101M | The Trichomonas vaginalis G3 whole genome shotgun |
| vaginalis | AbD serotec | monoclonal, | (WGS) project has the acession# |
| | The cell localization of the | Meridian | NZ_AAHC00000000. This version of the project(01) |
| | immunogen for this antibody | C65657M | has the accession# NZ_AAHC01000000, and consists |
| | is unknown. | monoclonal, | of sequences NZ_AAHC01000001- |
| | | Meridian | NZ_AAHC01075128. |
| | | C65675M | Length: 178,315,610 bp (unfinished). |
| | | monoclonal, | |
| | | Meridian | |
| | | C65526M | |
| | | monoclonal, | |
| | | Meridian | |
| | | PTV 65 | |
| | | polyclonal, | |
| | | HyTest | |
| | | Ab24317 | |
| | | Polyclonal, | |
| | | abcam | |
| Lactobacillus | | | 146 nucleotide sequences in CoreNucleotide Database, |
| crispatus | | | for example: gene for 16S rRNA, glyceraldehyde-3- |
| | | | phosphate dehydrogenase, S-layer proteins and 60 kDa |
| | | | chaperonin. |
| | | | 63 protein sequences, for example: phenolic acid |
| | | | decarboxylase, alpha-enolase, transposase, truncated |
| | | | PTS EIIC-like protein and surface layer protein. |
| Lactobacillus | | | 37 nucleotide sequences in CoreNucleotide Database, |
| jensenii | | | for example: gene for 16S ribosomal RNA, rpoA gene |
| | | | for RNA polymerase alpha subunit, pheS gene for |
| | | | phenylalanyl-tRNA synthase alpha subunit, ptsH gene, |
| | | | promoter region and 5′ UTR, rpsU gene, promoter |
| | | | region and 5′ UTR, 60 kDa heat shock protein, recA |
| | | | gene for recombinase A, mRNA for putative elongation |
| | | | factor Tu, 60 kDA chaperonin and 16S-23S rRNA |
| | | | intergenic spacer region and 23S rRNA gene 5' flanking |
| | | | region. |
| | | | 6 protein sequences, for example: RNA polymerase |
| | | | alpha subunit, phenylalanyl-tRNA synthase alpha |
| | | | subunit, 60 kDa heat shock protein, recombinase A, |
| | | | putative elongation factor Tu and 60 kDa chaperonin. |
| Lactobacillus | | | 261 nucleotide sequences in CoreNucleotide Database, |
| gasseri | | | for example: gene for 16S ribosomal RNA, 60 kDa |
| | | | chaperonin, rpoA gene for RNA polymerase alpha |
| | | | subunit, pheS gene for phenylalanyl-tRNA synthase |
| | | | alpha subunit, putative complement factor, acidocin |
| | | | LF221B, and putative immunity protein genes, recA |
| | | | gene for recombinase A, pbgal gene for phospho-beta- |
| | | | galactosidase, ATP synthase beta chain, aggregation |
| | | | promoting factor, gassericin T gene region, Gassericin |
| | | | A, phospho-beta-galactosidase and many sequences |
| | | | from patents. |
| | | | Genome sequences for Lactobacillus gasseri ATCC |
| | | | 33323, Length: 1,894,360 bp.3967 protein sequences, |
| | | | for example: putative immunity protein, acidocin |
| | | | LF221A, putative complemental factor, Lysin, Holin, |
| | | | phospho-beta-galactosidase, gassericin K7 B, putative |
| | | | ATP-dependent transport protein, aminopeptidase N, |
| | | | putative branched-chain amino acid transporter, beta- |
| | | | glucuronidase, putative regulatory protein, gassericin T, |
| | | | Gassericin A, ATP synthase alpha subunit, RNA |
| | | | polymerase alpha subunit, Ribosomal protein L34, |
| | | | RNase P protein component, Preprotein translocase |
| | | | subunit YidC, Predicted membrane protein, |
| | | | transcriptional regulator, Aggregation promoting |
| | | | factrelated surface protein and uncharacterized |
| | | | conserved secreted or membrane protein |

The present disclosure also incorporates by reference the technology disclosed for the use of SERS tags in assays including sandwich assays and lateral flow immunoassays (LFI). In particular, the following patents and pending applications are incorporated herein by reference; U.S. Pat. No. 6,514,767, Surface Enhanced Spectroscopy—Active Composite Nanoparticles; U.S. Pat. No. 7,192,778, Surface Enhanced Spectroscopy—Active Composite Nanoparticle; U.S. Patent App. Publication No. US-2005-0219509; Surface Enhanced Spectroscopy—Active Composite Nanoparticles; PCT Patent App. No. PCT/US07/61136 (unpublished), Lateral Flow Immunoassay with Encapsulated Detection Modality; PCT Patent Publication No. WO-2007/092941, SERS Nanotag Assays.

SERS allows detection of molecules attached to the surface of a single Raman-enhancing nanoparticle. A Raman-enhancing metal that has associated or bound to it a Raman-active molecule(s) is referred to as a SERS-active nanoparticle. Such SERS-active nanoparticles can have utility as optical tags. For example, SERS-active nanoparticles can be used in immunoassays when conjugated to an antibody against a target molecule of interest. If the target of interest is immobilized on a solid support, then the interaction between a single target molecule and a single nanoparticle-bound antibody could be detected by searching for the Raman-active molecule's unique Raman spectrum. Furthermore, because a single Raman spectrum (from 100 to 3500 cm⁻¹) can detect many different Raman-active molecules, different SERS-active nanoparticles can be used in multiplexed assay formats. In one embodiment, the disclosure provides for a single diagnostic for BV through the measurement of Lactobacillus. In an alternative embodiment, the disclosure provides for a multiplexed assay for BV, Candida, and Trichomonas, determining BV through the measurement of Lactobacillus.

The present disclosure also provides a lateral flow immunoassay (LFI) featuring encapsulated metal particles. Briefly, the encapsulated particles may use SERS nanotags as the detection modality. The use of encapsulated particles as a detection modality, in particular encapsulated SERS tags increases the sensitivity of an LFI prepared for visual reading and introduces the ability to obtain substantially more sensitive qualitative results or quantitative results through the analysis of a SERS spectrum read from an LFI prepared in accordance with the present embodiment. The use of SERS as detection modality also enhances the ability of an LFI device to be used for a multiplexed test. Other embodiments include LFI devices specifically configured to test vaginal samples, a reader for the detection and interpretation of a multiplexed assay and the hardware and software components used to implement the reader.

Note that throughout this application various citations are provided. Each of these citations is specifically incorporated herein by reference in its entirety.

### EXAMPLE 1

In one embodiment, Lactobacillus sequence will be used to determine antigenic regions and use this to raise antibodies. These antibodies will be used for quantitative measurements of Lactobacillus.

### EXAMPLE 2

In another embodiment, the sequences known for Lactobacillus are used to perform quantitative polymerase chain reaction for a determination of the amount of Lactobacillus.

### EXAMPLE 3

In one embodiment, a quantitative measurement of clinically relevant Lactobacilli is performed. This may include crispatus, jensenii, gasseri either independently, or with antibody/homologous cross reactive but specific reagents. As is known by one with skill in the art, a clinically relevant cut off value for the Lactobacillus measure via clinical trials and the use of statistical measures of relevance (i.e. ROC curves) are developed. Once the clinically relevant cut off value for Lactobacilli is established, a diagnostic test is performed, wherein the Lactobacilli of a patient is measured, and a diagnosis of BV is established based on a low clinically relevant Lactobacilli value.

### EXAMPLE 4

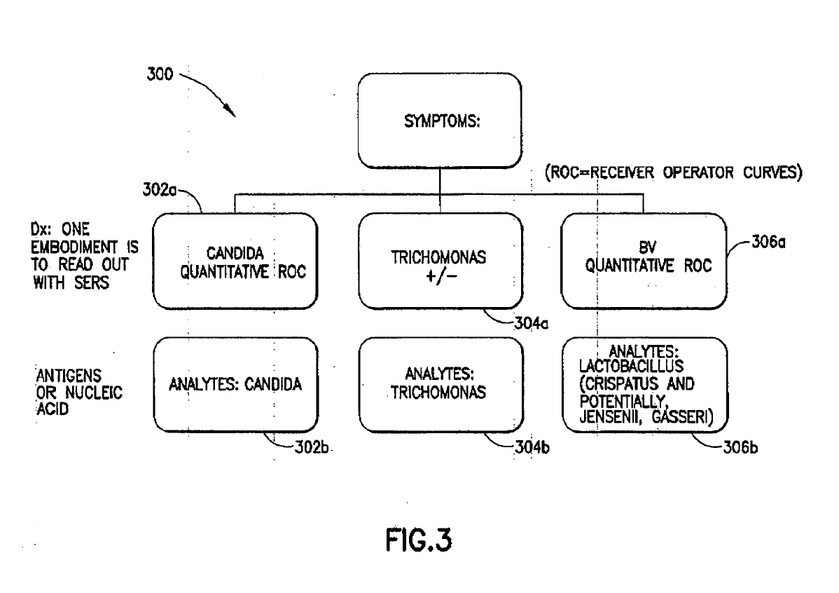

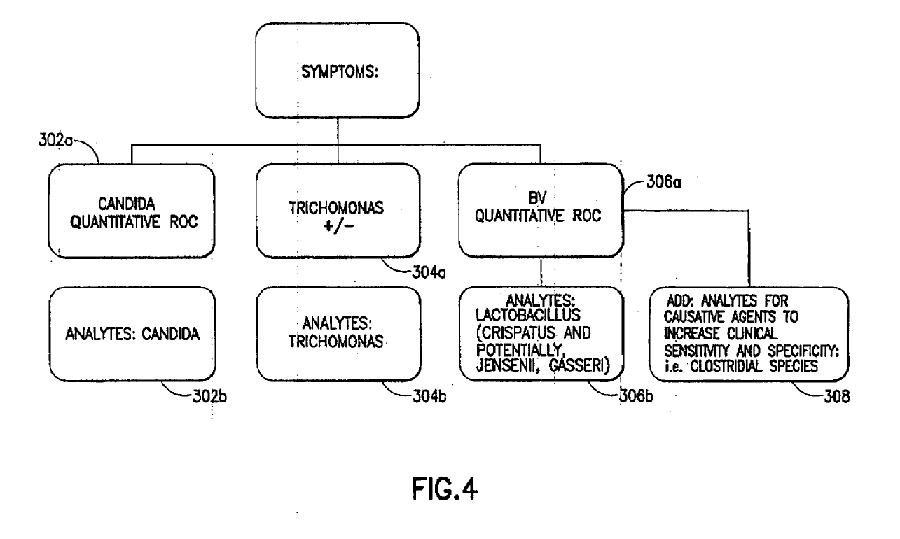

In one embodiment, the disclosure teaches a syndromic approach to vaginitis diagnosis. The three most common forms of infectious vaginitis in decreasing incidence are bacterial vaginosis, vulvovaginal candidiasis and trichomoniasis. As shown in FIG. 3, incorporating the three measures in one test 300 results in a very powerful test. Candida and Lactobacillus measures 302(a), 302(b), 304(a) and 304(b), respectively, are quantitative while the Trichomonas would be a test based on the presence or absence of Trichomonas 306(a), 306(b). As is known by one with skill in the art, a clinically relevant cut off value for the Lactobacillus measure via clinical trials and the use of statistical measures of relevance (i.e. ROC curves) are developed. Once the clinically relevant cut off value for Lactobacilli is established, a diagnostic test is performed, wherein the Lactobacilli of a patient is measured, and a diagnosis of BV is established based on a low clinically relevant Lactobacilli value. Similarly, for vulvovaginal candidiasis, clinical trials and the use of statistical measures of relevance (i.e. ROC curves) are developed. Once the clinically relevant cut off value is developed for Candida, diagnosis of vulvovaginal candidiasis is established based on a high clinically relevant Candida value. Trichomoniasis is diagnosed based on the presence of Trichomonas. In one embodiment a kit comprising the three tests based on LIF is envisioned using SERs tags for the relevant moieties. As shown in FIG. 4, an additional step 308 may selectively be implemented where analytes for causative agents to increase clinical sensitivity and specificity are added to the initial quantitative lactobacillus determination.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

1. A method for detecting the occurrence or non-occurrence of bacterial vaginosis in a patient comprising the steps of:
collecting a biological sample from a patient;
quantitatively measuring the presence of Lactobacillus; and
correlating the diminished presence of Lactobacillus with a diagnosis of bacterial vaginosis.

2. The method of claim 1 wherein said step of quantitatively measuring the presence of Lactobacillus comprises performing polymerase chain reaction for Lactobacillus.

3. The method of claim 1 wherein said step of quantitatively measuring the presence of Lactobacillus comprises performing a sandwich assay for Lactobacillus.

4. The method of claim 3 wherein said sandwich assay is a lateral flow immunoassay.

5. The method of claim 4 wherein said lateral flow immunoassay comprises SERS technology.

6. The method of claim 1 wherein said Lactobacillus is selected from the group consisting of Lactobacillus crispatus, Lactobacillus jensenii and Lactobacillus gasseri.

7. A method for the diagnosis infectious vaginitis comprising
collecting a biological sample from a patient;
measuring the presence of Lactobacillus, Candida and Trichomona; and
correlating said measurements;
wherein a diminished presence of Lactobacillus is indicative of bacterial vaginosis, an increased presence of Candida is indicative of vulvovaginal candidiasis and the presence of Trichomona is indicative of trichomoniasis.

8. The method of claim 7, wherein the measuring is performed in a single test kit.

9. The method of any of claims 7 or 8, wherein the measuring of Lactobacillus and Candida are quantitative measurements.

10. The method of any of claims 7 or 8, wherein said measuring is for the presence or absence of Trichomona.

11. The method of any of claims 7 or 8, wherein said measuring is performed comprising a lateral flow immunoassay.

12. A kit comprising an apparatus and instructions wherein the apparatus is a multiplex platform wherein testing for Lactobacillus, Candida and Trichomona is performed.

13. The method of claim 9, wherein said measuring is performed comprising a lateral flow immunoassay.

14. The method of claim 10, wherein said measuring is performed comprising a lateral flow immunoassay.
